(19) United States
(12) Patent Application Publication
(10) Pub. No.: US 2023/0368928 A1
(43) Pub. Date: Nov. 16, 2023

(54) EVENT-BASED DYNAMIC PATIENT COMMUNICATION

(71) Applicant: Brightree LLC, Peachtree Corners, GA (US)

(72) Inventors: Nupura KOLWALKAR, Cumming, GA (US); James Chang YI, Johns Creek, GA (US)

(21) Appl. No.: 18/180,309

(22) Filed: Mar. 8, 2023

Related U.S. Application Data
(60) Provisional application No. 63/341,342, filed on May 12, 2022.

(51) Int. Cl. G16H 80/00; G16H 10/60; G06F 40/186

(52) U.S. Cl. CPC G16H 80/00; G16H 10/60; G06F 40/186

(57) ABSTRACT

Techniques for improved dynamic transmission are provided. Occurrence of a defined event, of a plurality of defined events, is detected within a healthcare supply platform. A user corresponding to the occurrence of the defined event is identified, and a link to a customized webpage is generated based on a defined webpage template corresponding to the defined event. A transmission is generated based on a defined message template corresponding to the defined event, wherein the transmission includes the link to the customized webpage. The first transmission is transmitted to the user.

### CROSS-REFERENCE TO RELATED APPLICATIONS

This application claims priority to U.S. Provisional Patent Application No. 63/341,342, filed May 12, 2022, the entire content of which is incorporated herein by reference in its entirety.

### INTRODUCTION

Embodiments of the present disclosure relate to dynamic patient transmissions. More specifically, embodiments of the present disclosure relate to event-driven and dynamic communications.

In a wide variety of systems and platforms, reliable content provisioning and communication plays an important role in the operations of the system, as well as in improving user outcomes. For example, in healthcare settings, healthcare providers frequently engage with patients in order to monitor ongoing therapies, provide needed supplies and medications, and the like. However, in conventional systems, these transmissions are entirely manual. That is, the doctor or other healthcare professional must manually determine when to provide relevant content or other communications, and then must manually generate and transmit the content. As a result, the transmissions often fail to serve the underlying purpose (such as ensuring that users or patients are well informed). For example, the transmissions are often sent with a delay (e.g., after they are no longer useful) or are not sent at all (e.g., because the healthcare provider does not have time to prepare each such transmission, and/or because they simply forget to do so).

Improved systems and techniques to provide automated and dynamic transmissions are needed.

### SUMMARY

According to one embodiment presented in this disclosure, a method is provided. The method includes: detecting occurrence of a first defined event, of a plurality of defined events, within a healthcare supply platform; identifying a user corresponding to the occurrence of the first defined event; generating a link to a customized webpage based on a defined webpage template corresponding to the first defined event; generating a first transmission based on a defined message template corresponding to the first defined event, wherein the first transmission includes the link to the customized webpage; and transmitting the first transmission to the user.

According to one embodiment presented in this disclosure, a system is provided. The system comprises: a memory comprising computer-executable instructions; and one or more processors configured to execute the computer-executable instructions and cause the processing system to perform an operation comprising: detecting occurrence of a first defined event, of a plurality of defined events, within a healthcare supply platform; identifying a user corresponding to the occurrence of the first defined event; generating a link to a customized webpage based on a defined webpage template corresponding to the first defined event; generating a first transmission based on a defined message template corresponding to the first defined event, wherein the first transmission includes the link to the customized webpage; and transmitting the first transmission to the user.

According to one embodiment presented in this disclosure, a non-transitory computer-readable medium is provided, comprising computer-executable instructions that, when executed by one or more processors of a processing system, cause the processing system to perform an operation comprising: detecting occurrence of a first defined event, of a plurality of defined events, within a healthcare supply platform; identifying a user corresponding to the occurrence of the first defined event; generating a link to a customized webpage based on a defined webpage template corresponding to the first defined event; generating a first transmission based on a defined message template corresponding to the first defined event, wherein the first transmission includes the link to the customized webpage; and transmitting the first transmission to the user.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for event-based dynamic patient engagement.

FIG. 2 depicts an example user device and interface for event-based dynamic transmissions.

FIG. 3 depicts an example interface for defining event-based dynamic transmissions.

FIG. 4 is a flow diagram depicting an example method for providing dynamic event- based transmissions.

FIG. 5 is a flow diagram depicting an example method for using machine learning to predict resource usage and trigger dynamic patient interactions.

FIG. 6 is a flow diagram depicting an example method for generating dynamic transmission for event-based communication.

FIG. 7 is a flow diagram depicting an example method for authorizing and performing dynamic transmission.

FIG. 8 is a flow diagram depicting an example method for generating dynamic event-based webpages.

FIG. 9 is a flow diagram depicting an example method for generating event-based monitors to drive dynamic engagement.

FIG. 10 is a flow diagram depicting an example method for training machine learning models for resource prediction.

FIG. 11 is a flow diagram depicting an example method for training machine learning models based on resource usage data.

FIG. 12 is a flow diagram depicting an example method for training machine learning models based on therapy compliance data.

FIG. 13 is a flow diagram depicting an example method for generating event-based dynamic transmissions.

FIG. 14 depicts an example computing device configured to perform various aspects of the present disclosure.

Additional aspects of the present disclosure can be found in the attached appendix.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved event-based dynamic communications. In some embodiments, a variety of events within a healthcare platform can be defined (e.g., by a user or administrator). Upon the occurrence of such an event, in one embodiment, automated messages (e.g., text messages sent to a user's cell phone) can be generated (e.g., based on defined templates) and transmitted. This ensures that the users (e.g., patients) remain informed with the relevant information they need. Although some embodiments disclosed herein refer to healthcare platforms and patients as examples, aspects of the present disclosure are readily applicable to a wide variety of messaging, communication, transmission, or other information-providing systems. Similarly, although some aspects of the present disclosure refer to communication with patients, the term patients may generally include any user, including the patient themselves, a guardian or care provider for the patient, and the like.

Some embodiments of the present disclosure automate important communications to patients to streamline a variety of healthcare-related workflows, including therapeutic treatments, medication or healthcare product resupply, and the like. By leveraging a variety of paradigms such as email, text messaging, and direct push notifications, some embodiments of the present disclosure are able to reach more patients via more digital avenues in order to stay better connected and improve patient outcomes. For example, using aspects of the present disclosure, patients are more likely to have the information they need to continue their therapeutic journey, improving the eventual results (e.g., improving the probability of successful treatments, improving the time required to reach such success, and the like).

Additionally, in some embodiments, by dynamically predicting and responding to upcoming resupply needs (e.g., when the patient will need more equipment, such as new tubes or filters for a continuous positive airway pressure (CPAP) machine), the system is able to improve the functionality of the healthcare platform (e.g., by providing additional services and reducing manual effort), as well as improving the healthcare outcomes for the patients. In at least one embodiment, the system can utilize machine learning to predict or detect when patients are at risk of failing their therapy (e.g., if they are going to stop using their CPAP machine). Such machine learning-based events can trigger further content and actions, thereby reducing patient attrition and improving patient outcomes.

In some embodiments, the automatically-generated transmission can further include links or pointers to dynamic and customized webpages that are generated specifically for the context of the automated transmission. For example, the system may generate a mobile first landing page that is specifically designed for ease of use on mobile devices, and transmit the link to this page via text message. These mobile first pages can provide application-like experiences (e.g., simulating or closely mirroring a dedicated app on the user's phone) without requiring them to download an application, register or create an account, or even remember login credentials. By removing these barriers to use, the system is able to drive enhanced user interaction and engagement with reduced effort by the patient. Further, because the content can be custom-delivered via a webpage, the computational expenses of the mobile device are reduced. For example, because the user need not download an application, the needed bandwidth by the user device is reduced. Similarly, because the user accesses the custom content via a link to the custom webpage, the amount of memory and storage needed on the user device is reduced, as compared to conventional systems. In this way, aspects of the present are able to provide transmissions and functionality that meets or surpasses conventional solutions, while reducing or eliminating a variety of computational burdens on the user devices.

### Example Environment for Event-Based Dynamic Patient Engagement

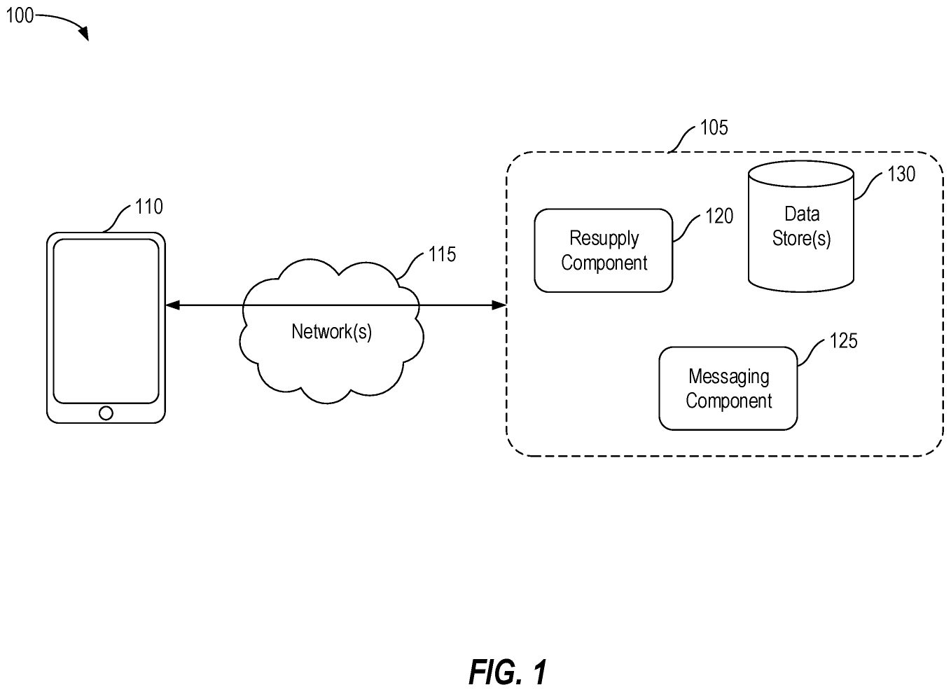

FIG. 1 depicts an example environment 100 for event-based dynamic patient engagement.

In the illustrated example, a healthcare platform 105 is communicatively coupled with a user device 110 via a network 115. Although the illustrated example includes a healthcare platform 105, in embodiments, a wide variety of platforms and systems may implement aspects of the present disclosure. Generally, any system that provides messaging, communications, transmissions, or other content to users may use embodiments of the present disclosure to provide improved and dynamic communications.

Although a single healthcare platform 105 is depicted, in embodiments, there may be any number of such platforms or systems. The healthcare platform 105 is generally representative of one or more systems or devices that collectively or separately provide one or more aspects of healthcare to users. For example, the healthcare platform 105 may be used to provide and monitor therapeutic treatments (such as CPAP or BiPAP treatments) to patients, to provide engagement and updated information, and the like. In the illustrated example, the healthcare platform 105 has a variety of associated components, including a resupply component 120, a messaging component 125, and one or more data stores 130.

In the healthcare platform 105, the resupply component 120 may generally be used to detect and/or predict resupply needs, detect and/or predict patient attrition, provide or trigger resource resupply, and the like. For example, the resupply component 120 may use machine learning to evaluate user data (e.g., stored in the data stores 130) in order to predict when a user is likely to stop using a therapeutic device (e.g., a CPAP), as discussed in more detail below. The resupply component 120 can generally be implemented using hardware, software, or a combination of hardware and software, and its operations may be combined or distributed across any number of components and systems. Additionally, though depicted as operating within the healthcare platform 105 for conceptual clarity, in some embodiments, the resupply component 120 operates as a third-party service.

The messaging component 125 is generally configured to generate, transmit, and/or receive messages, content, transmissions, or other data to and from user devices 110. In one embodiment, the messaging component 125 may generate transmissions using defined transmission templates, as discussed in more detail below, and transmit these messages to the user devices 110 via one or more networks. For example, when relevant therapy information becomes available, the messaging component 125 may identify the corresponding patient(s), generate a customized text message for the patient, embed a custom and dynamic link to a customized webpage generated for the new information, and transmit the message via a cellular network.

The messaging component 125 can generally be implemented using hardware, software, or a combination of hardware and software, and its operations may be combined or distributed across any number of components and systems. Additionally, though depicted as operating within the healthcare platform 105 for conceptual clarity, in some embodiments, the messaging component 125 operates as a third-party service.

Generally, the resupply component 120 and/or messaging component 125 can identify or detect the occurrence of a variety of defined events within the healthcare platform 105, and generate corresponding messages appropriately. For example, a user or administrator may define specific triggering criteria that are used to initiate a transmission. Upon detecting such an event, the messaging component 125 can generate and transmit an appropriate message (e.g., using a template, and/or user-specific data in the data stores 130) to the affected or relevant user device(s) 110, as discussed in more detail below.

In the illustrated example, the user device 110 is depicted as a smartphone. However, the user device 110 may correspond to a wide variety of devices and systems, including tablets, desktop or laptop computers, wearable devices (e.g., smart watches), and the like. Although a single user device 110 is depicted for conceptual clarity, there may be multiple user devices 110. Each user device 110 is generally associated with or otherwise linked to a corresponding user or patient. In some embodiments, a single patient may have multiple user devices 110 (e.g., a smartphone, a tablet, and a smart watch). The user device 110 generally corresponds to a computing device used, by a user (e.g., a patient), to receive and/or interact with digital content, as discussed below in more detail.

As illustrated, the user device 110 and healthcare platform 105 are communicatively linked via one or more networks 115. The networks 115 generally correspond to any communication link that enables content to be transmitted between the user device 110 and healthcare platform 105. Although a single network 115 is depicted for conceptual clarity, there may be any number and variety of networks 115. In at least one embodiment, the user device 110 and healthcare platform 105 are connected via multiple networks. For example, the user device and healthcare platform 105 may be configured to transmit and receive text messages via a cellular network, as well as to transmit and receive data via the Internet. In this way, the healthcare platform 105 can provide content via one network 115 (e.g., transmitting a custom text message), and the user device 110 can respond via the same network (e.g., by texting back) or a different network (e.g., by following a custom link, included in the text message, to a webpage).

In an embodiment, the messaging component 125 may be configured to automatically provide relevant content for a wide variety of events and circumstances with respect to each patient. For example at a referral or intake stage (e.g., when the patient is referred to a specialist or other healthcare provider), the healthcare platform 105 may identify this referral as an event, and generate one or more messages based on predefined templates (e.g., to welcome the new patient, to ask them to complete or provide information needed for intake, to estimate or indicate future expected costs, to discuss insurance or payment options, to provide status updates, to set up appointments or healthcare resource deliveries, and the like.

Similarly, the healthcare platform 105 may detect events such as needed or upcoming resource shipments and generate dynamic content related to when the delivery is expected, who the delivery driver or provider will be, troubleshooting or informational content for the new equipment, facilitating returns, and the like. As another example, the healthcare platform 105 may generate information related to when resupply is upcoming, use automated messages to confirm that resupply is needed or desired by the patient, and the like.

As further examples, the healthcare platform 105 may detect events related to appointments, and generate corresponding messages such as to remind the patient about the appointment, allow for re-scheduling, providing information or details about the specifics of the appointment (e.g., about the therapist or other healthcare provider the patient will be meeting with), enabling check-in prior to the appointment, facilitating virtual meetings, and the like.

In some embodiments, further events (and corresponding generated messages) can relate to billing, collections, therapy compliance, and the like. For example, the system may use detected events to drive transmissions related to auto-pay, invoicing, insurance updates, expiring forms of payment, contact information, therapy compliance concerns (e.g., when the patient may stop using the therapy), and the like.

Generally, each event can have user-defined triggering criteria for a wide variety of variables, parameters, or other occurrences within (or external to) the healthcare platform 105. In an embodiment, upon detection of any such event, the healthcare platform 105 automatically identifies the relevant patient(s), generates a context-specific message (e.g., using a defined message template for the event), optionally generates a custom (mobile-first) webpage to be linked in the message (e.g., using a webpage template and information specific to the current event and context), and transmits it to the patient(s). This enables rapid and efficient information allocation, improves patient outcomes, reduces computational expense on the user devices 110 and healthcare platform 105, reduces manual effort and error, and generally improves the overall system functionality significantly.

### Example Interfaces for Event-Based Dynamic Transmissions

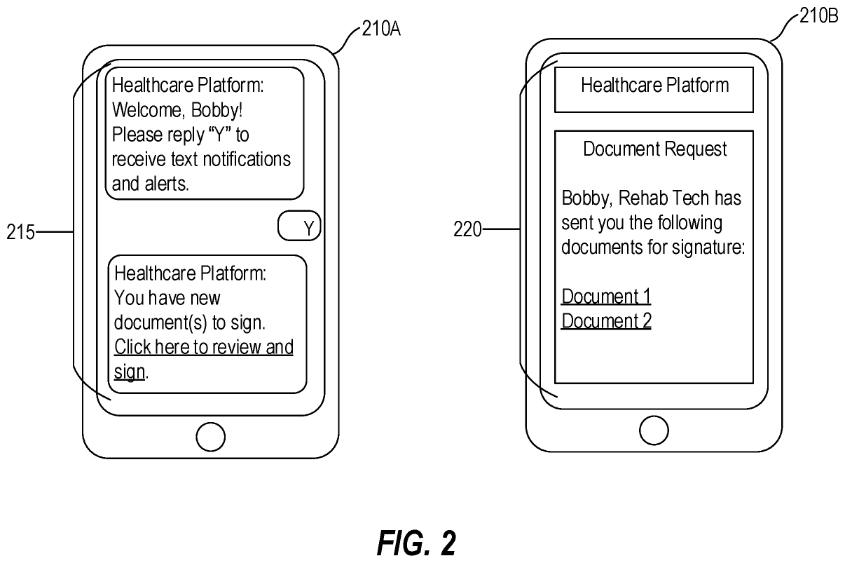

FIG. 2 depicts an example user device and interface for event-based dynamic transmissions.

In the illustrated example, the user device 210 may correspond to the user device 110 of FIG. 1. The illustrated example depicts a user device 210A and a user device 210B, which may correspond to two distinct user devices, or to the same user device at different times. As illustrated, each user device 210 is outputting, via a graphical user interface (GUI), content that was automatically provisioned by a healthcare platform (such as the healthcare platform 105 of FIG. 1).

In the illustrated example, the user device 210A is displaying a text message conversation 215. Specifically, the text conversation 215 relates to an onboarding, intake, or referral event detected by the healthcare platform. As illustrated, the first message was generated, by the healthcare platform, using a predefined messages template, and was then transmitted via text (e.g., via a cellular network) to the user device 210A. Using the message template, the healthcare platform is able to use a standardized message format, and insert or append context-specific data (e.g., from data stores 130 of FIG. 1) for the specific patient and/or event. For example, as illustrated, the first message indicates the name of the healthcare platform or provider (e.g., “Healthcare Platform”), followed by a specific greeting generated based on the receiving patient (e.g., “Welcome Bobby!”). That is, the message may have been generated using a template such as “[Provider]: Welcome, [Patient]!”, where [Provider] and [Patient] are replaced using context-specific information for the event.

As illustrated, the first transmission further includes a request that the user opt-in to text notifications and alerts by responding “Y”. In some embodiments, such authorization or validation can be selectively requested, by the healthcare platform, depending on the context of the specific event and patient. For example, some implementations may require the patient to opt-in or approve text messaging only for specific types of events, at specific times (e.g., only at the intake stage), and the like.

In the illustrated example, when the patient replies by opting in, the healthcare platform detects this response as another event, and accordingly generates a corresponding message to return. Specifically, this event may be identified based on the opt-in response, as well as the surrounding context (e.g., the current intake event, or the next-step in the intake). In the illustrated example, the healthcare platform determines that the next event is a signature request. In response, the healthcare platform generates and transmits another text message (using another event-specific template) to indicate that there are documents needing the patient's signature. In this message, as illustrated, the healthcare platform also includes a dynamically-generated link to a customized webpage for the request/event. That is, the healthcare platform includes a link for the user, allowing them to quickly access, review, and sign the needed documents.

The user device 210B is displaying a mobile first webpage 220 that was generated, by the healthcare platform, using a predefined webpage template that corresponds to the original event. For example, the webpage 220 can be generated using a template (e.g., a “signature request”) template that corresponds to the event that triggered the original message (e.g., the second message displayed by the user device 210A). That is, when an event occurs, the healthcare platform may generate not only a custom and dynamic message based on the event, but also generate a link to a custom webpage corresponding to the event.

As discussed above, the webpage 220 can generally be used to provide an app-like experience, without requiring the user to download an application, log-in, or otherwise provide any credentials or context. That is, because the webpage is accessed via the custom link (e.g., provided via text), the healthcare platform can understand the context of the request when the user selects the link (e.g., when the patient uses it to request the webpage from the healthcare platform). In the illustrated example, the webpage 220 also includes an indication of the provider (“Healthcare Platform”), as well as information relating to the specific user (“Bobby”), the relevant entities for the request (“Rehab Tech”), and the needed response from the patient (signing “Document 1” and “Document 2”).

By using the custom-generated mobile first webpage 220, the healthcare platform allows the user to review and sign the documents while reducing bandwidth and memory footprint on the user device 210B (e.g., because the user need not download or install an application), as well as reducing the manual effort needed by the patient (e.g., by not requiring them to sign in). Further, as the webpage 220 is generated dynamically for the specific patient only when the event occurs (and because other users cannot access the information using stolen credentials, as no such credential sexist), security and user privacy is improved.

Although not included in the illustrated examples, in some embodiments, the healthcare platform can optionally include a link to log-in or register with the healthcare platform (in the text messages and/or in the webpage). Such a login system may be used for some event types (e.g., for payment information), for some patients (e.g., when the patient prefers to use credentials), and the like.

### Example Interface for defining Event-Based Dynamic Transmissions

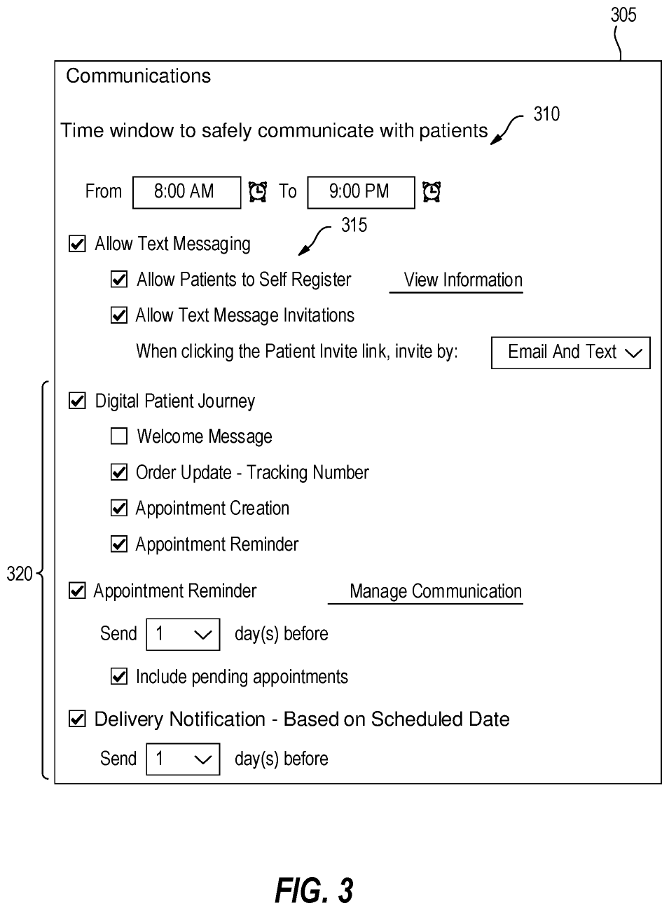

FIG. 3 depicts an example interface 305 for defining event-based dynamic transmissions. The interface 305 may be used by a user or administrator of a healthcare platform, such as the healthcare platform 105 of FIG. 1, to define the relevant events and messages used by the platform to interact with users (e.g., patients).

In the illustrated example, the interface 305 includes a variety of options and configurations for the healthcare platform. For example, at 310, the user can configure the time(s) when messages should be transmitted to patients. In the illustrated example, messages (e.g., text messages) are enabled between the hours of 8:00 AM and 9:00 PM. In one embodiment, if an event is detected outside of this window (e.g., at 10:00 PM), the healthcare platform can queue the event for future processing. This may include, for example, queueing the event itself, such that when the window re-opens (at 8:00 AM) the healthcare platform can generate and transmit a corresponding message. In at least one embodiment, the healthcare platform can proceed to generate the message(s) and/or webpage(s) for the events that occur outside of the allowable window, and queue these messages for transmission when the window opens. This can spread the computational load of message generation across the non-peak hours (e.g., overnight), thereby reducing the workload on the healthcare platform during the indicated allowable messaging hours. This improves the functionality and operations of the system (e.g., by reducing or eliminating computational bottlenecks during the day, when other workloads are likely present, and moving them to overnight times).

At 315, the healthcare platform can further use interface 305 to selectively enable and disable a variety of messaging options, such as whether text messages are enabled (e.g., as compared to email messages, push messages, and the like), as well as whether patients will be allowed to self-register (e.g., by following a link in the text).

As illustrated in block 320, the interface 305 then provides a variety of options for specific events to be monitored in the healthcare platform. Specifically, the illustrated example depicts a set of events (each indicated using a checkbox), where each event corresponds to a predefined configuration (e.g., predefined triggering criteria and/or message content). For example, a user or administrator may have defined the events for future use by other users, administrators, or systems. In some embodiments, the interface 305 can additionally or alternatively allow users to manually create new events, as discussed in more detail below.

In the illustrated example, the block 320 includes a set of events that fall under a “Digital Patient Journey” type, an “Appointment Reminder” type, and a “Delivery Notification” type. Although three types are depicted for conceptual clarity, there may be any number of groups of events. Additionally, in some aspects, the events are not grouped into broader headings. In the illustrated example, each event can be individually toggled (e.g., using the checkbox associated with each event), and the broader groups of events can be toggled to control the underlying events (e.g., where toggling off the “Digital Patient Journey” checkbox will disable messages for all of the events within that category).

As discussed above, each individual event generally has a set of triggering criteria, as indicated using a textual summary or name in the interface 305. For example, the “Welcome Message” event may trigger at patient intake, and the “Order Update-Tracking Number” event may trigger when a tracking number becomes available for a healthcare resource order (such as when the patient has ordered new equipment or medication).

As illustrated, some of the events can additionally have further configurations available via the interface 305. For example, the “appointment reminder” event allows the user to define when the reminder(s) should be sent (e.g., one day before the appointment).

In some embodiments, the interface 305 can be used to control event-based messaging across the healthcare platform. That is, the interface 305 may enable and disable specific event monitoring for all patients. In at least one embodiment, the event-based monitoring can be individually tailored for specific individuals. For example, a first patient may wish to receive messages triggered based on appointment creation, while another patient does not. In some embodiments, the interface 305 enables such patient-specific event monitoring to be configured as well.

### Example Method for Providing Dynamic Event-Based Transmissions

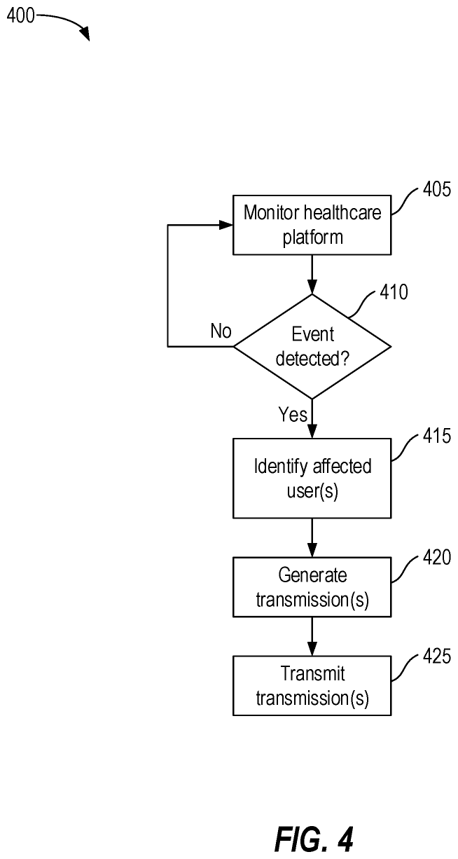

FIG. 4 is a flow diagram depicting an example method 400 for providing dynamic event-based transmissions. In some embodiments, the method 400 is performed by a healthcare platform, such as the healthcare platform 105 of FIG. 1. For example, the method 400 may be implemented by a messaging component of a healthcare platform, such as messaging component 125 of FIG. 1.

At block 405, the messaging component monitors a healthcare platform for a set of defined events. As discussed above, monitoring for events can generally include monitoring any variety of data, such as new patient referrals, order shipments, and the like. In some embodiments, monitoring for events includes monitoring one or more databases or other data stores (such as data store 130 of FIG. 1) for updates. For example, each time a new row is added to a “patient” table in a database, the messaging component may determine that a “new patient” event has occurred. In some embodiments, monitoring for events includes receiving push notifications about new events occurring. For example, a shipping service may indicate, to the messaging component, that an order has shipped. In some embodiments, monitoring the healthcare platform can include monitoring events occurring within the platform (e.g., new information available for a specific patient), as well as events occurring outside of the platform that are otherwise related to the platform (such as an appointment with a third party, or delivery of a medical device).

At block 410, the messaging component determines whether occurrence of any events was detected during the monitoring. As discussed above, the messaging component can generally use a defined set of events, each with corresponding triggering criteria indicating when the event is considered to have occurred. If no events have occurred, the method 400 returns to block 405 to continue monitoring the platform.

If, at block 410, the messaging component determines that an event occurred, the method 400 continues to block 415, where the messaging component identifies any users (also referred to as patients) that are affected by, implicated in, or otherwise associated with or relevant to the detected event. Generally, the relevant user(s) may be identified based at least in part on the event itself. In some embodiments, the relevant users are identified using criteria defined in the event itself. For example, for “order shipment” events, the relevant users may be the intended recipient(s) of the order. Similarly, for a “signature request” event, the relevant user may be person whose signature is required (or their caregiver or custodian).

At block 420, the messaging component can then generate a transmission, for the identified user(s), based on the detected event. In some embodiments, as discussed below in more detail, generating the transmission includes retrieving a defined message or transmission template associated with the detected event, identifying and retrieving relevant information to include (e.g., to replace placeholders in the template), such as the user's name, and the like. In some embodiments, as discussed below in more detail, generating the transmission includes generating a custom link to a customized webpage based on the event.

At block 425, the messaging component transmits the generated transmission(s). For example, as discussed above, the messaging component may transmit the content as a text message to the phone number(s) of the identified user(s). In some embodiments, prior to transmitting the message, the messaging component can confirm that the transmission is appropriate (e.g., that the current time is within the window of defined acceptable times, which may be global or user-specific). If the current time is outside of the desired window, the messaging component may place the generated transmission in a queue, to be transmitted when appropriate.

In this way, the messaging component can use the method 400 to drive dynamic, customized, event-based transmissions to users (e.g., patients), providing up-to-date information in an efficient and timely manner. As discussed above, this can reduce manual effort, increase information flow, reduce error, improve user outcomes, reduce computational expense, and generally improve the operations of the healthcare platform as a whole.

### Example Method for Using Machine Learning to Predict Resource Usage and Trigger Dynamic Patient Interactions

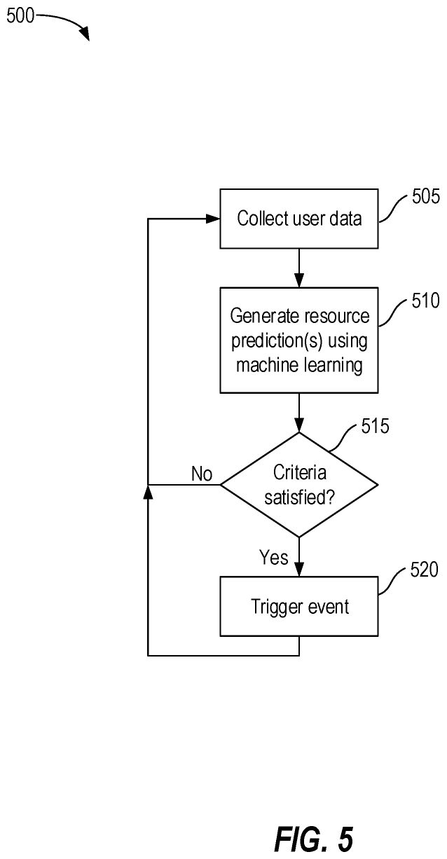

FIG. 5 is a flow diagram depicting an example method 500 for using machine learning to predict resource usage and trigger dynamic patient interactions. In some embodiments, the method 500 is performed by a healthcare platform, such as the healthcare platform 105 of FIG. 1. For example, the method 500 may be implemented by a messaging component of a healthcare platform, such as messaging component 125 of FIG. 1, and/or by a resupply component, such as resupply component 120 of FIG. 1. The method 500 generally provides additional detail for specific event types that are trigger by machine learning models, rather than by a rules-based event criteria.

At block 505, the healthcare platform collects user data in the platform (e.g., from data stores 130 of FIG. 1). For example, as discussed below in more detail, the healthcare platform may evaluate a variety of user data such as the patient's specific characteristics (e.g., diagnoses, ongoing therapies, order history, therapy compliance or state data, and the like). Although the method 500 depicts evaluation of user data for a single specific user, in embodiments, the method 500 may be used to process data for any number of patients or users.

At block 510, the healthcare platform processes the user data using one or more machine learning models to generate one or more resource predictions. As used herein, a resource prediction is generally a forecast, prediction, or estimation for future healthcare resource usage of the user. As used herein, healthcare resource usage may refer to the ordering of, consumption of, or otherwise need for healthcare resources by the user. As used herein, healthcare resources can generally include a wide variety of items, such as medications, supplements, equipment (e.g., CPAP machines), portions or parts of equipment (e.g., new hoses or masks for a CPAP machine), and the like.

In embodiments, the resource prediction can generally include predictions as to resources that will be ordered or needed (e.g., based on predicting that the user will run out of supplies at a point in the future), predicting that resources will not be ordered (e.g., because the user is at risk of no long following the therapeutic treatment and therefore will not order more supplies), and the like.

At block 515, the healthcare platform determines whether one or more defined criteria are satisfied. In some embodiments, multiple criteria may be used to trigger distinct events. For example, a first criteria may be satisfied if the resource predictions indicate that the user will request a resupply at a defined point or window in the future (e.g., within the next week). As another example, the criteria may be satisfied if the resource predictions indicate that the user will need a resupply (e.g., because their current supply is expected to expire, be consumed, or otherwise need replacement), but that the user is not likely to request the resupply (e.g., because they are no longer following the treatment plan).

If no criteria are satisfied, the method 500 returns to block 505 to collect new user data for additional processing (e.g., for another user, or for a subsequent window or period in time). If one or more criteria are satisfied, the method 500 continues to block 520, where the healthcare platform triggers one or more events based on the generated predictions. In some embodiments, the specific triggered event(s) are determined based on the predictions themselves, and/or based on the criteria that were satisfied.

For example, if the predictions and/or criteria indicate that the user is expected to request additional resupply in the next week, an event corresponding to a resupply order, prompt, or reminder may be triggered. Continuing this example, the healthcare platform may use this triggered event to generate a custom message (e.g., using the method 400 of FIG. 4) to indicate, to the user, which supplies will be needed, how they can order or approve the request, and the like.

As another example, if the predictions and/or criteria indicate that the user is likely to stop the treatment program (or has already stopped complying), the triggered event may include an “attrition” event or other event used to identify, prevent, and/or reverse patient attrition. For example, the healthcare platform may use the event to generate a custom message with information, suggestions, offers of assistance, and the like in order to bring the patient back into compliance. This can improve patient healthcare outcomes substantially.

After the event(s) are triggered, at block 520, the method 500 returns to block 505 to process new data (e.g., for a new user, or for the same user in a future window of time).

### Example Method for Generating Dynamic Transmissions for Event-Based Communication

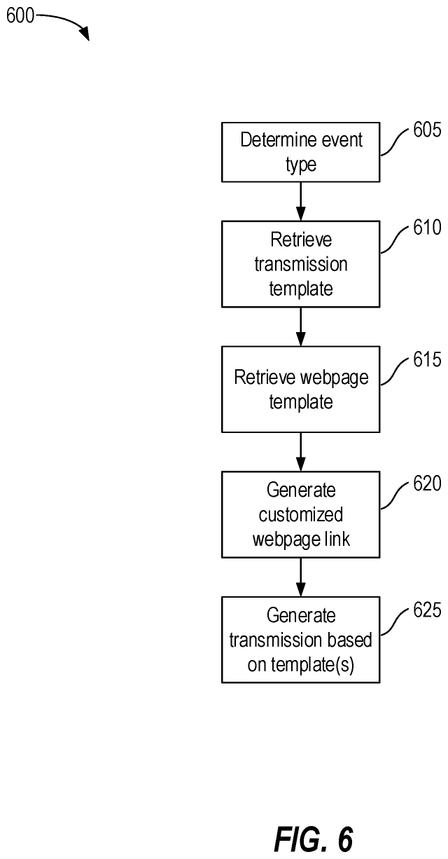

FIG. 6 is a flow diagram depicting an example method 600 for generating dynamic transmissions for event-based communication. In some embodiments, the method 600 is performed by a healthcare platform, such as the healthcare platform 105 of FIG. 1. For example, the method 600 may be implemented by a messaging component of a healthcare platform, such as messaging component 125 of FIG. 1, and/or by a resupply component, such as resupply component 120 of FIG. 1. In some embodiments, the method 600 provides additional detail for block 420 of FIG. 4, where custom transmissions are generated based on detected events.

At block 605, the healthcare platform determines the type of the detected event. For example, the healthcare platform may determine whether the detected event is an “order shipment” event, a “new patient” event, and the like.

At block 610, the healthcare platform retrieves a transmission template based on the determined event type. As discussed above, each event type may have a corresponding defined message or transmission template that is used to generate transmissions when the event type is detected. For example, a “welcome” event may be triggered when a new patient enrolls or is referred to the healthcare platform, and the corresponding message template may include “[Provider]: Welcome, [Patient]! We are looking forward to working with you on [Therapy]!” In some embodiments, the transmission templates can include natural language text, to be included in the message, as well as one or more placeholders where user-specific (or event-specific) information can be placed. For example, the healthcare platform may insert information such as the specific user associated with the event, the timing of the event, the affected services or therapies, and the like.

At block 615, the healthcare platform retrieves a webpage template based on the determined event type. As discussed above, some or all of the event types may have a corresponding defined webpage template that is used to generate a webpage (e.g., a mobile first page) for the event type. For example, a “document signature” event may be triggered when a provider needs the patient to sign a document, and the corresponding website template may include natural language text and/or formatting to render a webpage that provides any additional detail, and allows the user to review and sign the documents. In some embodiments, the webpage templates can include natural language text, to be included on the page, as well as one or more placeholders where user-specific (or event-specific) information can be placed. For example, the healthcare platform may insert information such as the name of the specific user associated with the event, links to the document(s) to be reviewed, and the like.

At block 620, the healthcare platform generates a customized webpage link based on the determined event type and webpage template. In some embodiments, the healthcare platform generates the link based on the information, indicated in the webpage template, needed to provide context for the request. For example, the webpage link may include contextual information (e.g., identifying the user, the event, and the like). When the user uses the link to retrieve the webpage, the healthcare platform can use this context to generate a custom webpage, using the webpage template, that is specifically designed for the detected event. In some embodiments, the healthcare platform can generate the webpage as soon as the event occurs (e.g., before transmitting the message). In other embodiments, the healthcare platform first generates the customized link based on the template. When the user subsequently uses the link, the healthcare platform can dynamically generate a webpage based on the context of the link and the corresponding template.

At block 625, the healthcare platform generates the transmission based on the message template and webpage link. For example, as discussed above, the healthcare platform may fill any placeholders in the transmission template based on the specific context of the detected event, and insert or append the custom webpage link into the message. In this way, the user can review the customized transmission message, and optionally use the custom link, to access their specific information without the need for further registration or providing of credentials.

### Example Method for Authorizing and Performing Dynamic Transmission

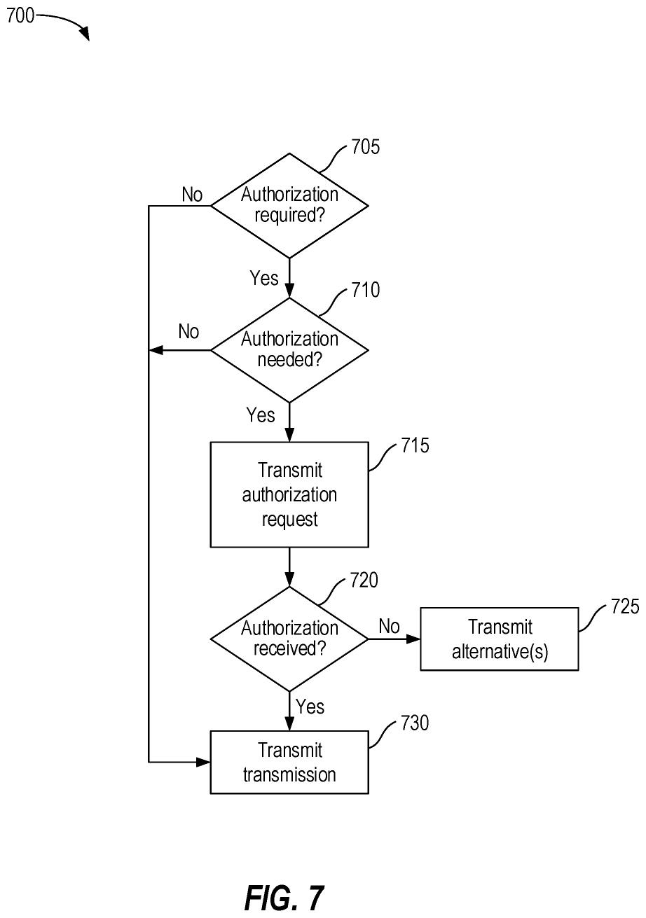

FIG. 7 is a flow diagram depicting an example method 700 for authorizing and performing dynamic transmission. In some embodiments, the method 700 is performed by a healthcare platform, such as the healthcare platform 105 of FIG. 1. For example, the method 700 may be implemented by a messaging component of a healthcare platform, such as messaging component 125 of FIG. 1, and/or by a resupply component, such as resupply component 120 of FIG. 1. In some embodiments, the method 700 provides additional detail for block 425 of FIG. 4, where generated transmissions are transmitted.

At block 705, the healthcare platform determines whether authorization is needed for the generated transmission. For example, as discussed above, some messages (or event types) may require user approval before the message can be delivered via text message. As another example, the healthcare platform may require that all users approve text message notifications at the intake process.

If, at block 705, the healthcare platform determines that approval is not needed (e.g., because the user already approved, or because the specific type of message that is being sent does not require approval), the method 700 continues to block 730, where the healthcare platform transmits the transmission (e.g., as a text message over a cellular network). If, at block 705, the healthcare platform determines that authorization is needed, the method 700 continues to block 710.

At block 710, the healthcare platform determines whether the required authorization is still needed, or has already been acquired. For example, the healthcare platform may determine that the type of event requires user authorization, but that the user has already approved or authorized the specific type of message.

If authorization is not needed (e.g., because the user already provided it), the method 700 continues to block 730, where the healthcare platform transmits the transmission (e.g., as a text message using a cellular network). If, at block 710, the healthcare platform determines that authorization is required and needed (e.g., it has not been obtained for the specific user and/or event type), the method 700 continues to block 715.

At block 715, the healthcare platform transmits an authorization request to the user. For example, as discussed above, the healthcare platform may transmit a message (e.g., a text message) asking the user to reply “Y” to opt-in to text alerts, “N” to opt-out, and/or “help” to get more information or help.

At block 720, the healthcare platform determines whether user authorization has been received. Continuing the above example, for instance, the healthcare platform may determine whether the user has replied “Y” or some other assent to the authorization request (e.g., via text message). If authorization was received, the method 700 continues to block 730, where the healthcare platform transmits the original content to the user (e.g., as a text message via a cellular network).

If authorization was declined (e.g., the user replied “N”), the method 700 continues to block 725. At block 725, the healthcare platform can transmit the content via one or more alternatives. For example, rather than use text messaging, the healthcare platform may transmit the content via email, via direct message, as a push notification using an application on the user's device, and the like.

### Example Method for Generating Dynamic Event-Based Webpages

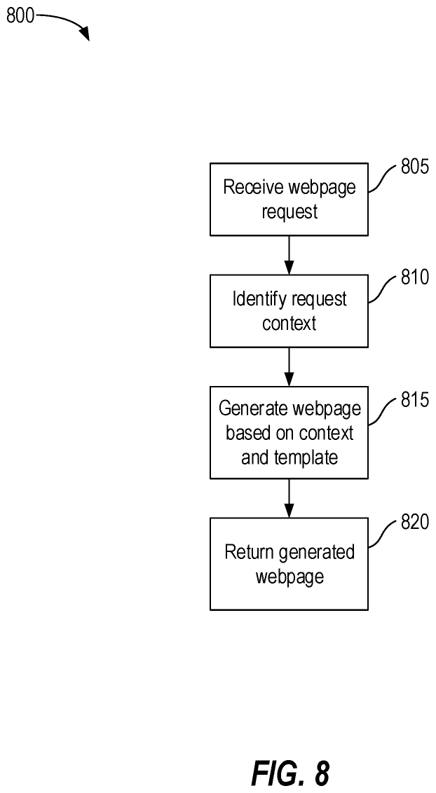

FIG. 8 is a flow diagram depicting an example method 800 for generating dynamic event-based webpages. In some embodiments, the method 800 is performed by a healthcare platform, such as the healthcare platform 105 of FIG. 1. For example, the method 800 may be implemented by a messaging component of a healthcare platform, such as messaging component 125 of FIG. 1, and/or by a resupply component, such as resupply component 120 of FIG. 1.

At block 805, the healthcare platform receives a webpage request from a user device (e.g., user device 110 of FIG. 1, and/or user device 210 of FIG. 2). In some embodiments, as discussed above, the webpage is requested using a customized link that was transmitted, by the healthcare platform, to the user device in response to detection of an event on the healthcare platform. For example, if the healthcare platform determines that a healthcare resource (e.g., a new CPAP machine) is scheduled to be delivered the healthcare platform may generate and transmit a message, to the patient, notifying them of the delivery, providing information for the delivery person (e.g., a brief profile, a picture, and the like), and including a link for more information.

At block 810, the healthcare platform identifies the context of the webpage request. In some embodiments, as discussed above, the context is embedded within the link itself. For example, the link may include a path such as “www.fakehealthcarewebsite.com/PATIENT/EVENT,” where “PATIENT” and “EVENT” identify the specific patient and event, respectively. As another example, the link may include the context appended as a parameter or keyword on the path, such as “www.fakehealthcarewebsite.com?patient=PATIENT&event=EVENT,” where “PATIENT” and “EVENT” are the specific patient and detected event, respectively.

In some embodiments, the healthcare platform can determine the context of the request based at least in part on the identity of the requestor. For example, the healthcare platform may identify the user device that used the link to request the webpage (e.g., the MAC address or other unique identifier), and use this identifier to lookup the corresponding patient and the desired context.

At block 815, the healthcare platform generates a customized and dynamic webpage based on the determined context and a defined website template. In one such embodiment, as discussed above, each event (or event type) may have a corresponding template (e.g., an HTML template) that defines the arrangement of elements on the page, based at least in part on the event-specific elements that will be included. For example, a “signature request” template may include a header identifying the healthcare platform, a section with text where information such as the patient name, signature requestor, document summary, and the like can be inserted, and one or more links to the documents.

In an embodiment, generating the page based on the context includes inserting the context/patient-specific information wherever needed in the template. For example, the patient name, document description, and the like can be inserted into the webpage before it is returned to the requesting patient.

In at least one embodiment, as discussed above, the webpage template uses a mobile-first design. That is, the template may be specifically designed for viewing/interaction using a mobile device (e.g., a smartphone), as opposed to a desktop or other computing device. As the user is likely accessing it via their phone (e.g., by tapping a link included in a text message), the healthcare platform can therefore provide a seamless experience that effectively simulates a dedicated application on the user's device without requiring the user to actually download, install, or sign in to any such application.

At block 820, the healthcare platform then returns the generated customized webpage to the patient. As discussed above, this seamless provisioning of dynamic and customized information allows the user (e.g., the patient, or a caregiver or guardian) to receive the relevant information at any point in time in a way that reduces computational expense and memory footprint on the user device (e.g., because the user need not download or maintain an application), minimizes bandwidth usage (e.g., because the user need not download an application or authenticate themselves once connected), and generally improves the functioning of the healthcare platform, user device, and overall networking and communication system.

### Example Method for Generating Event-Based Monitors to Drive Dynamic Engagement

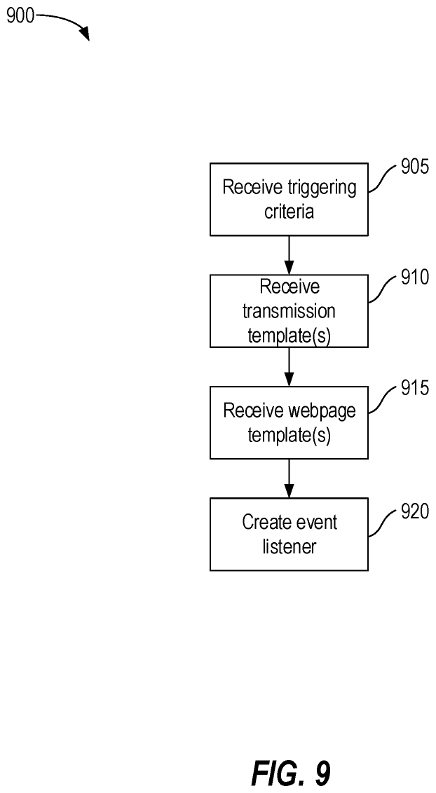

FIG. 9 is a flow diagram depicting an example method 900 for generating event-based monitors to drive dynamic engagement. In some embodiments, the method 900 is performed by a healthcare platform, such as the healthcare platform 105 of FIG. 1. For example, the method 900 may be implemented by a messaging component of a healthcare platform, such as messaging component 125 of FIG. 1, and/or by a resupply component, such as resupply component 120 of FIG. 1. In at least one embodiment, the method 900 is used to define triggering events and responses (e.g., using the interface 305 of FIG. 3).

At block 905, the healthcare platform receives defined triggering criteria (e.g., from a user or administrator). As discussed above, the triggering criteria generally indicates one or more events, variables, values, or other data that should be used to trigger an event/response. For example, the triggering criteria may include receiving a shipping or delivery notification from a shipping entity, identifying a new or updated row or table in a database, and the like. Generally, the triggering criteria can include specified events within the healthcare platform, events occurring outside of the platform (such as shipping, where an alert may be sent to the healthcare platform), and the like.

At block 910, the healthcare platform receives one or more transmission templates, defined by a user or administrator, to be used when the triggering criteria are satisfied. For example, as discussed above, the transmission templates may include natural language text explaining the event/providing information or updates to the receiving user. In some embodiments, the templates include one or more placeholders where context-specific information (such as the name of the patient, or detail for the specific event occurrence) can be inserted, as discussed above.

At block 915, the healthcare platform optionally receives one or more webpage templates, defined by the user or administrator, to be used if a link is embedded in the transmission. As discussed above, some transmissions may not include such links. For such events, a webpage template is not needed. In some embodiments, as discussed above, the webpage template can specify the layout or arrangement of elements, as well as the content of such elements, based on the specific event. When the receiving user uses the link to request the webpage, the healthcare platform can use the provided template to generate a customized webpage including the relevant information or context for the user.

At block 920, the healthcare platform creates an event listener for the event. For example, using the provided triggering criteria, the healthcare platform can configure an event listener to monitor the healthcare platform to detect when the criteria are satisfied and the event is triggered. As discussed above, this allows the healthcare platform to dynamically identify and respond to the defined set of events using customized and event-specific responses (e.g., transmitted via text message) to patients.

### Example Method for Training Machine Learning Models for Resource Prediction

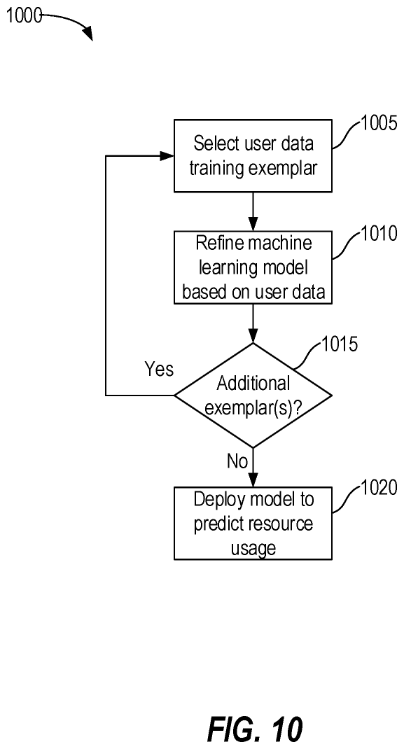

FIG. 10 is a flow diagram depicting an example method 1000 for training machine learning models for resource prediction. In some embodiments, the method 1000 is performed by a healthcare platform, such as the healthcare platform 105 of FIG. 1. For example, the method 1000 may be implemented by a messaging component of a healthcare platform, such as messaging component 125 of FIG. 1, and/or by a resupply component, such as resupply component 120 of FIG. 1.

In some embodiments, the method 1000 is performed by one or more dedicated training systems. In one such embodiment, a training system (which may be included within the healthcare platform, or may be a separate system) may be used to train and/or refine the machine learning models. These trained models may then be deployed on the same system (e.g., within the healthcare platform), and/or on one or more other systems.

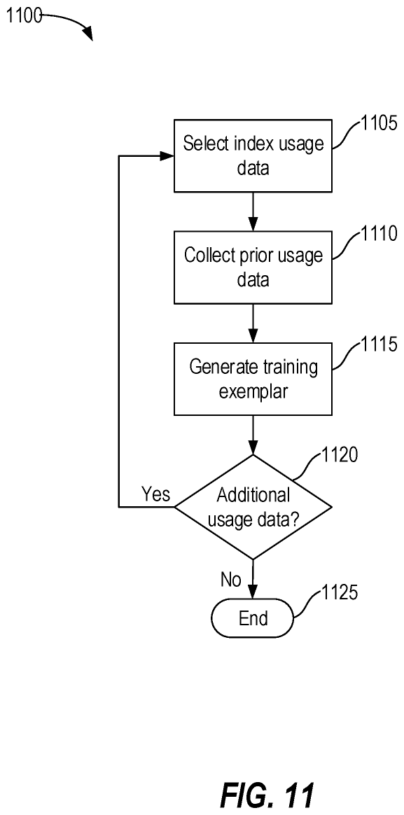

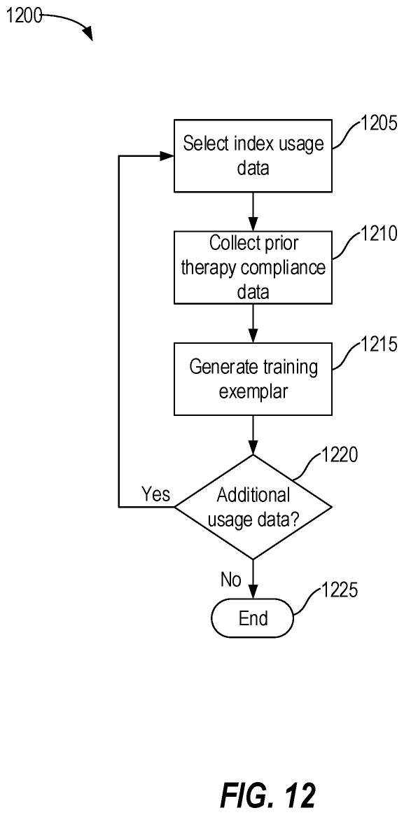

At block 1005, the training system selects a training exemplar, the exemplar including user data. In some embodiments, the specific contents of the user data may vary depending on the particular implementation and purpose of the machine learning model. For example, as discussed below in more detail with reference to FIG. 11, the user data may include data relating to resource usage by a user (e.g., order history). As another example, as discussed below in more detail with reference to FIG. 12, the user data my include therapy compliance and/or therapy state data (e.g., records relating to how often the user complies with or uses the therapy, such as how regularly they use their CPAP machine).

Generally, the user data training exemplar includes a set of user data from one or more windows of time, as well as a corresponding label indicating resource usage, by the user, during or after the one or more windows of time. For example, the user data may include characteristics of the user during a given month, and the label may indicate the healthcare resources that were used, ordered, requested, or otherwise consumed by the user during that month or during the subsequent month.

At block 1010, the training system refines a machine learning model based on the selected exemplar. For example, the training system may provide the user data as input to the model in order to generate a resource prediction (e.g., predicting whether the user will request additional resources, based on the user data). The output prediction can then be compared against the known label of the exemplar (e.g., the actual resource usage) in order to generate a loss, which can be used to refine the parameters of the model to generate more accurate predictions. In this way, the model can learn to predict resource usage for any user, based on the current and/or historical characteristics of the user.

At block 1015, the training system determines whether there is at least one additional training exemplar remaining for the training process. If so, the method 1000 returns to block 1005 to select another record. Generally, this selection can be performed according to any suitable criteria, including randomly. In some embodiments, in addition to or instead of determining whether additional exemplars remain, the training system may determine whether other termination criteria are satisfied, such as a maximum amount of time or computational resources spent training, a minimum model accuracy (determining using text exemplars), and the like.

Although the illustrated process depicts an iterative and sequential training process using each exemplar to individually refine the model (e.g., using stochastic gradient descent), in some aspects, the model may be trained using batches of exemplars (e.g., using batch gradient descent).

If no further exemplars remain (or if other termination criteria are satisfied), the method 1000 continues to block 1020, where the training system deploys the model to predict resource usage during runtime. For example, as discussed above, the training system may use the trained model(s) to predict whether users will request more healthcare resources (e.g., within a defined period of time), whether users will actually need resources (e.g., to determine whether the user is at risk of stopping the therapy), and the like. In some embodiments, the output of the models, during runtime, can be used to trigger events and corresponding messages, such as discussed above with reference to FIG. 5.

In some embodiments, the method 1000 can be used to provide initial training of the machine learning model(s). In at least one embodiment, the method 1000 can additionally or alternatively be used to provide model refinement or fine-tuning. In one such embodiment, during runtime, the training system or healthcare platform can monitor resource usage to generate new training exemplars. For example, the training system may periodically or continuously collect user data for one or more users, label or tag the data based on the user's resource request history and/or therapy attrition, and then store these exemplars for future training or refinement of the models. This can ensure that the models are refined using newly-collected data and therefore maintain high accuracy for the system.

### Example Method for Training Machine Learning Models based on Resource Usage Data

FIG. 11 is a flow diagram depicting an example method 1100 for training machine learning models based on resource usage data. In some embodiments, the method 1100 is performed by a healthcare platform, such as the healthcare platform 105 of FIG. 1. For example, the method 1100 may be implemented by a messaging component of a healthcare platform, such as messaging component 125 of FIG. 1, and/or by a resupply component, such as resupply component 120 of FIG. 1.

In some embodiments, the method 1100 is performed by one or more dedicated training systems. In one such embodiment, a training system (which may be included within the healthcare platform, or may be a separate system) may be used to train and/or refine the machine learning models. These trained models may then be deployed on the same system (e.g., within the healthcare platform), and/or on one or more other systems. In at least one embodiment, the method 1100 is performed to generate training data (e.g., training exemplars) used to train and/or refine machine learning models, as discussed above with reference to FIG. 10.

At block 1105, the training system selects an index set of usage data, corresponding to a user or patient, to be used as the label or target output for the training. For example, the training system may select, determine, or otherwise collect the user's healthcare resource usage (e.g., consumption or order history) for a given point or window in time, and use this usage as the index or target data.

At block 1110, the training system collects one or more sets of prior usage data for the user, from one or more windows of time prior to the index usage data. For example, if the index usage data corresponds to the user's orders or resupply requests during a given month, the prior data may include the user's orders or resupply requests during one or more prior months. In an embodiment, this prior data is used as the input portion of the exemplar. That is, when training the model, the prior data may be used as input, and the resulting generated prediction can be compared against the index data.

At block 1115, the training system generates a training exemplar based on the index and prior usage data. As discussed above, this training exemplar can thereafter be used to train or refine a machine learning model to predict future resource usage or requests (e.g., future resupply orders) based on current and/or past resource usage or requests (e.g., previous resupply orders).

At block 1120, the training system determines whether there is additional usage data that can be used to generate a training exemplar. For example, the training system may determine whether there is additional data for the same user (e.g., for a different time period), data from a different user, and the like. If so, the method 1100 returns to block 1105. If not, the method 1100 terminates at block 1125, and the generated exemplars can be used to train or refine one or more machine learning models.

### Example Method for Training Machine Learning Models based on Therapy Compliance Data

FIG. 12 is a flow diagram depicting an example method 1200 for training machine learning models based on therapy compliance data. In some embodiments, the method 1200 is performed by a healthcare platform, such as the healthcare platform 105 of FIG. 1. For example, the method 1200 may be implemented by a messaging component of a healthcare platform, such as messaging component 125 of FIG. 1, and/or by a resupply component, such as resupply component 120 of FIG. 1.

In some embodiments, the method 1200 is performed by one or more dedicated training systems. In one such embodiment, a training system (which may be included within the healthcare platform, or may be a separate system) may be used to train and/or refine the machine learning models. These trained models may then be deployed on the same system (e.g., within the healthcare platform), and/or on one or more other systems. In at least one embodiment, the method 1100 is performed to generate training data (e.g., training exemplars) used to train and/or refine machine learning models, as discussed above with reference to FIG. 10.

At block 1205, the training system selects an index set of usage data, corresponding to a user or patient, to be used as the label or target output for the training. For example, the training system may select, determine, or otherwise collect the user's healthcare resource usage (e.g., consumption or order history) for a given point or window in time, and use this usage as the index or target data.

At block 1210, the training system collects prior therapy compliance data for the user associated with the index usage data. For example, if the usage data relates to an ongoing therapy such as use of a CPAP machine, the training system may collect data relating to the user's use of the CPAP therapy. This may include, for example, the (average) number of hours per day/night that the user has used it, the (average) number of days per week that the user uses the CPAP, and the like. In some embodiments, the compliance data can include other state data, such as the amount of mask leak reported (by the user or automatically by the CPAP machine). Generally, the therapy compliance data can include any information relating to whether and how much the user is using or complying with the therapy.

At block 1215, the training system generates a training exemplar based on the index usage data and the prior compliance/therapy state data. In this way, the training exemplar can thereafter be used to train or refine a machine learning model to predict future resource usage or requests (e.g., future resupply orders) based on current and/or past therapy compliance. For example, based on how often, how long, or how well the user is complying with the therapy, the model can learn to predict whether the user will stay on the therapy, request more resources, and the like.

At block 1220, the training system determines whether there is additional usage data that can be used to generate a training exemplar. For example, the training system may determine whether there is additional data for the same user (e.g., for a different time period), data from a different user, and the like. If so, the method 1200 returns to block 1205. If not, the method 1200 terminates at block 1225, and the generated exemplars can be used to train or refine one or more machine learning models.

### Example Method for Generating Event-Based Dynamic Transmissions

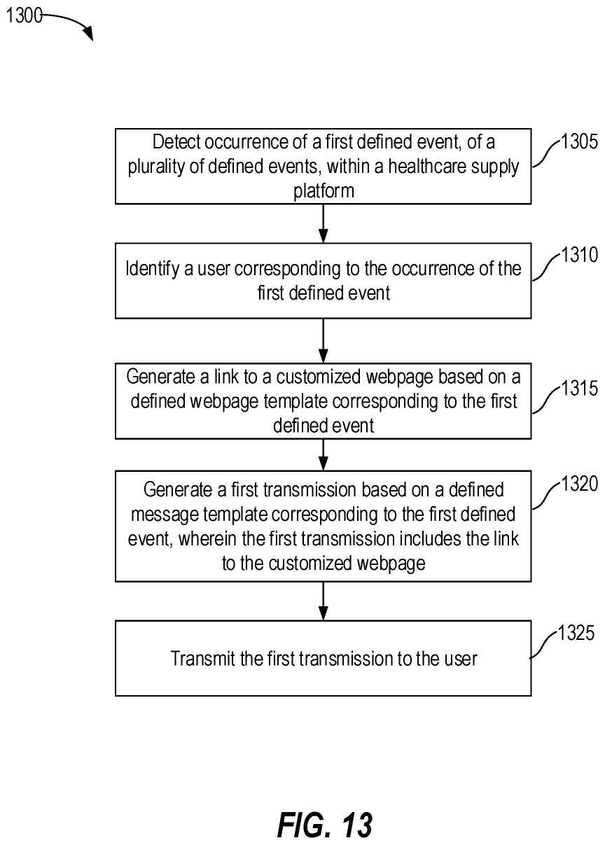

FIG. 13 is a flow diagram depicting an example method 1300 for generating event-based dynamic transmissions. In some embodiments, the method 1300 is performed by a healthcare platform, such as the healthcare platform 105 of FIG. 1. For example, the method 1300 may be implemented by a messaging component of a healthcare platform, such as messaging component 125 of FIG. 1, and/or by a resupply component, such as resupply component 120 of FIG. 1.

At block 1305, occurrence of a first defined event, of a plurality of defined events, is detected within a healthcare supply platform.

In some aspects, detecting occurrence of the first defined event comprises predicting that additional healthcare resources will be used, at a future time, for an ongoing therapy used by the user.

In some aspects, predicting that additional healthcare resources will be used comprises processing user data associated with the user using a machine learning model trained to predict healthcare resource usage.

In some aspects, the user data comprises historical healthcare resource request from the user.

In some aspects, the user data comprises historical therapy data indicating a status of the user with respect to the ongoing therapy.

At block 1310, a user corresponding to the occurrence of the first defined event is identified.

At block 1315, a link to a customized webpage is generated based on a defined webpage template corresponding to the first defined event.

In some aspects, the customized webpage includes a link allowing the user to create or sign into a user account for the healthcare supply platform.

At block 1320, a first transmission is generated based on a defined message template corresponding to the first defined event, wherein the first transmission includes the link to the customized webpage.

At block 1325, the first transmission is transmitted to the user.

In some aspects, the first transmission indicates the additional healthcare resources and prompts the user to request the additional healthcare resources.

In some aspects, the method further comprises, prior to transmitting the first transmission to the user: transmitting, to the user, a request for authorization to provide the first transmission, and receiving, from the user, authorization to provide the first transmission.

In some aspects, transmitting the first transmission comprises sending a text message to a cellular device of the user.

### Example User System for Improved Data Services

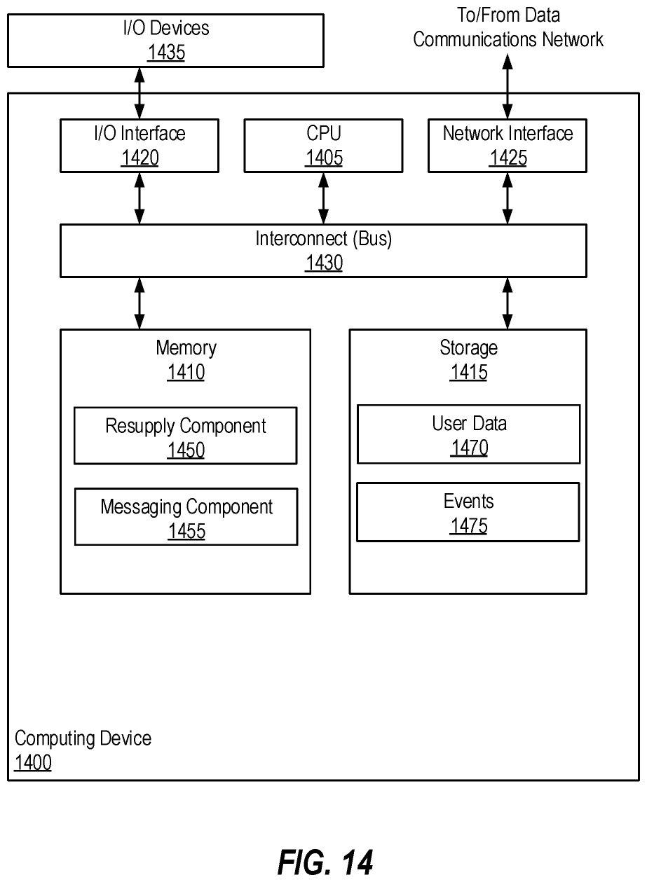

FIG. 14 depicts an example computing device 1400 configured to perform various aspects of the present disclosure. Although depicted as a physical device, in embodiments, the computing device 1400 may be implemented using virtual device(s), and/or across a number of devices (e.g., in a cloud environment). In one embodiment, the computing device 1400 corresponds to one or more systems in a healthcare platform, such as the healthcare platform 105 of FIG. 1.

As illustrated, the computing device 1400 includes a CPU 1405, memory 1410, storage 1415, a network interface 1425, and one or more 110 interfaces 1420. In the illustrated embodiment, the CPU 1405 retrieves and executes programming instructions stored in memory 1410, as well as stores and retrieves application data residing in storage 1415. The CPU 1405 is generally representative of a single CPU and/or GPU, multiple CPUs and/or GPUs, a single CPU and/or GPU having multiple processing cores, and the like. The memory 1410 is generally included to be representative of a random access memory. Storage 1415 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1435 (such as keyboards, monitors, etc.) are connected via the I/O interface(s) 1420. Further, via the network interface 1425, the computing device 1400 can be communicatively coupled with one or more other devices and components (e.g., via a network, which may include the Internet, local network(s), and the like). As illustrated, the CPU 1405, memory 1410, storage 1415, network interface(s) 1425, and I/O interface(s) 1420 are communicatively coupled by one or more buses 1430.

In the illustrated embodiment, the memory 1410 includes a resupply component 1450 and a messaging component 1455, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1410, in embodiments, the operations of the depicted components (and others not illustrated) may be implemented using hardware, software, or a combination of hardware and software.

In one embodiment, the resupply component 1450 may correspond to the resupply component 120 of FIG. 1. The resupply component 1450 may be used to predict patient resupply needs and/or therapy attrition (e.g., using machine learning), as discussed above. For example, the resupply component 1450 may process user data (e.g., therapy data and/or prior healthcare resource data) to generate predicted future resource usage and/or compliance. The messaging component 1455 may correspond to the messaging component 125 of FIG. 1. The messaging component 1455 may generally be used to detect occurrence of events, and generate corresponding messages or transmissions, as discussed above. For example, the messaging component 1455 may detect defined events, retrieve corresponding transmission templates, generate appropriate (customized) transmissions, and transmit them (e.g., via text message) to users (e.g., patients).

In the illustrated example, the storage 1415 includes user data 1470 and events 1475. In one embodiment, the user data 1470 may include attributes or characteristics of the users or patients, such as their prior resource usage or order history, their compliance status or behavior on the therapy, biographical or demographic information, and the like. The events 1475 can generally include defined triggering criteria and/or templates for one or more events, as discussed above, that are monitored by the system. Although depicted as residing in storage 1415, the user data 1470 and events 1475 may be stored in any suitable location, including memory 1410.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., messaging component 120 of FIG. 1) or related data available in the cloud. For example, the messaging component could execute on a computing system in the cloud and automatically generate dynamic transmissions based on detected events. In such a case, the messaging component could use context-specific information and defined templates to generate content, and store the templates and/or messages at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: detecting occurrence of a first defined event, of a plurality of defined events, within a healthcare supply platform; identifying a user corresponding to the occurrence of the first defined event; generating a link to a customized webpage based on a defined webpage template corresponding to the first defined event; generating a first transmission based on a defined message template corresponding to the first defined event, wherein the first transmission includes the link to the customized webpage; and transmitting the first transmission to the user.

Clause 2: The method of Clause 1, wherein detecting occurrence of the first defined event comprises predicting that additional healthcare resources will be used, at a future time, for an ongoing therapy used by the user.

Clause 3: The method of any one of Clauses 1-2, wherein the first transmission indicates the additional healthcare resources and prompts the user to request the additional healthcare resources.

Clause 4: The method of any one of Clauses 1-3, wherein predicting that additional healthcare resources will be used comprises processing user data associated with the user using a machine learning model trained to predict healthcare resource usage.

Clause 5: The method of any one of Clauses 1-4, wherein the user data comprises historical healthcare resource request from the user.

Clause 6: The method of any one of Clauses 1-5, wherein the user data comprises historical therapy data indicating a status of the user with respect to the ongoing therapy.

Clause 7: The method of any one of Clauses 1-6, wherein the customized webpage includes a link allowing the user to create or sign into a user account for the healthcare supply platform.

Clause 8: The method of any one of Clauses 1-7, the method further comprising, prior to transmitting the first transmission to the user: transmitting, to the user, a request for authorization to provide the first transmission; and receiving, from the user, authorization to provide the first transmission.

Clause 9: The method of any one of Clauses 1-8, wherein transmitting the first transmission comprises sending a text message to a cellular device of the user.

Clause 10: A system, comprising: a memory comprising computer-executable instructions; and one or more processors configured to execute the computer-executable instructions and cause the processing system to perform a method in accordance with any one of Clauses 1-9.

Clause 11: A system, comprising means for performing a method in accordance with any one of Clauses 1-9.

Clause 12: A non-transitory computer-readable medium comprising computer-executable instructions that, when executed by one or more processors of a processing system, cause the processing system to perform a method in accordance with any one of Clauses 1-9.

Clause 13: A computer program product embodied on a computer-readable storage medium comprising code for performing a method in accordance with any one of Clauses 1-9.

What is claimed is:

1. A method, comprising:
detecting occurrence of a first defined event, of a plurality of defined events, within a healthcare supply platform;
identifying a user corresponding to the occurrence of the first defined event;
generating a link to a customized webpage based on a defined webpage template corresponding to the first defined event;
generating a first transmission based on a defined message template corresponding to the first defined event, wherein the first transmission includes the link to the customized webpage; and
transmitting the first transmission to the user.

2. The method of claim 1, wherein detecting occurrence of the first defined event comprises predicting that additional healthcare resources will be used, at a future time, for an ongoing therapy used by the user.

3. The method of claim 2, wherein the first transmission indicates the additional healthcare resources and prompts the user to request the additional healthcare resources.

4. The method of claim 2, wherein predicting that additional healthcare resources will be used comprises processing user data associated with the user using a machine learning model trained to predict healthcare resource usage.

5. The method of claim 4, wherein the user data comprises historical healthcare resource request from the user.

6. The method of claim 4, wherein the user data comprises historical therapy data indicating a status of the user with respect to the ongoing therapy.

7. The method of claim 1, wherein the customized webpage includes a link allowing the user to create or sign into a user account for the healthcare supply platform.

8. The method of claim 1, the method further comprising, prior to transmitting the first transmission to the user:
transmitting, to the user, a request for authorization to provide the first transmission; and
receiving, from the user, authorization to provide the first transmission.

9. The method of claim 1, wherein transmitting the first transmission comprises sending a text message to a cellular device of the user.

10. A non-transitory computer-readable medium comprising computer-executable instructions that, when executed by one or more processors of a processing system, cause the processing system to perform an operation comprising:
detecting occurrence of a first defined event, of a plurality of defined events, within a healthcare supply platform;
identifying a user corresponding to the occurrence of the first defined event;
generating a link to a customized webpage based on a defined webpage template corresponding to the first defined event;
generating a first transmission based on a defined message template corresponding to the first defined event, wherein the first transmission includes the link to the customized webpage; and
transmitting the first transmission to the user.

11. The non-transitory computer-readable medium of claim 10, wherein detecting occurrence of the first defined event comprises predicting that additional healthcare resources will be used, at a future time, for an ongoing therapy used by the user.

12. The non-transitory computer-readable medium of claim 11, wherein the first transmission indicates the additional healthcare resources and prompts the user to request the additional healthcare resources.

13. The non-transitory computer-readable medium of claim 11, wherein predicting that additional healthcare resources will be used comprises processing user data associated with the user using a machine learning model trained to predict healthcare resource usage.

14. The non-transitory computer-readable medium of claim 13, wherein the user data comprises at least one of:
historical healthcare resource request from the user, or
historical therapy data indicating a status of the user with respect to the ongoing therapy.

15. The non-transitory computer-readable medium of claim 10, the operation further comprising, prior to transmitting the first transmission to the user:
transmitting, to the user, a request for authorization to provide the first transmission; and
receiving, from the user, authorization to provide the first transmission.

16. A system, comprising:
a memory comprising computer-executable instructions; and
one or more processors configured to execute the computer-executable instructions and cause the system to perform an operation comprising:
detecting occurrence of a first defined event, of a plurality of defined events, within a healthcare supply platform;
identifying a user corresponding to the occurrence of the first defined event;
generating a link to a customized webpage based on a defined webpage template corresponding to the first defined event;
generating a first transmission based on a defined message template corresponding to the first defined event, wherein the first transmission includes the link to the customized webpage; and
transmitting the first transmission to the user.

17. The system of claim 16, wherein detecting occurrence of the first defined event comprises predicting that additional healthcare resources will be used, at a future time, for an ongoing therapy used by the user.

18. The system of claim 17, wherein predicting that additional healthcare resources will be used comprises processing user data associated with the user using a machine learning model trained to predict healthcare resource usage.

19. The system of claim 18, wherein the user data comprises at least one of:
historical healthcare resource request from the user, or
historical therapy data indicating a status of the user with respect to the ongoing therapy.

20. The system of claim 16, the operation further comprising, prior to transmitting the first transmission to the user:
transmitting, to the user, a request for authorization to provide the first transmission; and
receiving, from the user, authorization to provide the first transmission.
